(19) United States
(12) Patent Application Publication
(10) Pub. No.: US 2007/0270584 A1
(43) Pub. Date: Nov. 22, 2007

(54) Process for the Preparation of Steroidal Carbothioic Acid Derivatives and Intermediates

(76) Inventors: Trond Loevli, Sofiemyr (NO); Anne-Mette Nygaard, Jar (NO); Bjoern Reitstoen, Oslo (NO); Magny Fivelstad, Oslo (NO)

Correspondence Address: CANTOR COLBURN, LLP, 55 GRIFFIN ROAD SOUTH, BLOOMFIELD, CT, 06002, US

(21) Appl. No.: 10/552,118

(22) PCT Filed: Apr. 2, 2004
(86) PCT No.: PCT/DK04/00242

§ 371 Date: Apr. 13, 2007

(30) Foreign Application Priority Data
Apr. 4, 2003 (EP) 03007756.4
Mar. 19, 2004 (DK) 2004 00449

(51) Int. Cl. C07J 43/00

(52) U.S. Cl. 540/111; 552/611; 540/110

(57) ABSTRACT

A method for the conversion of steroidal 17β-carboxylic acids to c carbothioic acid and esters thereof such as fluticasone propionate.

The present invention relates to a novel process for the conversion of steroidal carboxylic acids to carbothioate derivatives such as fluticasone propionate via novel intermediates.

### BACKGROUND OF THE INVENTION

In the preparation of 17β-carbothioic acids, sulphur has generally been introduced under anhydrous conditions; either by the use of hydrogensulfide (U.S. Pat. No. 4,335,121, U.S. Pat. No. 4,578,221) or by employing the salt of hydrogensulfide, generated in situ from the reaction between sodium hydride and hydrogensulfide (U.S. Pat. No. 4,188,385, U.S. Pat. No. 4,335,121, U.S. Pat. No. 4,578,221).

In US2002/0133032 Abbott claim a process towards fluticasone propionate starting from flumethasone. The process consists of 5 steps, of which the final two can be performed as a one-pot reaction, and the product is obtained in an overall yield of 51%. Abbott also claims that a 6α-chloro-9α-fluoro-impurity present in commercial grade flumethasone necessitates an elaborate purification of flumethasone prior to use.

In the preparation of fluticasone propionate, the introduction of the CH₂F-moiety has been accomplished by the reaction of the caribothioic metal salt with a dihalomethane derivative, preferably a fluorohalomethane. Glaxo first made use of bromochloromethane, followed by halogen exchanges (U.S. Pat. No. 4,335,121). They later moved on to bromofluoromethane (WO02088167, WO02100879, WO0212265, WO0212266), first mentioned by Phillips et al. (J. Med. Chem., 1994, 37, 3717-3729). The Israeli company Chemagis (IL109656) utilised both bromo- and chlorofluoromethane. Chlorofluoromethane was later used by Abbott as well (US2002/0133032).

### SUMMARY OF INVENTION

The problem to be solved by the present invention is to provide a new method for the preparation of steroidal carbothioic acid and derivatives thereof such as fluticasone propionate, especially a method in which a number of the relevant method steps may be performed as a continuous one-pot synthesis. This denotes a method where relevant synthetic steps may be performed ill situ without change of solvent or isolation of the individual intermediates.

The present invention provides a method which comprises A) reacting a steroidal carboxylic acid or a salt thereof with a coupling agent alone or in conjunction with a coupling enhancer; and B) reacting the product of step A) with a nucleophilic agent comprising a sulfur atom.

The solution is based on the identification by the present inventors that by employing novel in situ generated esters, such as 17β-carboxy-imidazolyl-, succinimidyl- or triazolyl esters of formula (III), as intermediates, an increased threshold against competing hydrolysis reactions was achieved. The reduced level of hydrolysis further raises the efficiency regarding the formation of the end steroidal carbothioate product and removes the need to work under strictly anhydrous conditions. The preferred steroidal carbothioate end products are defined by formula (I) herein. When R₁₀ of formula (I) is a fluoromethyl group this represents fluticasone propionate.

The intermediate (such as an ester of formula (III)) is very suitable for use in a method for the conversion of a steroidal carboxylic acid to a steroidal carbothioic acid or a carbothioate. As explained above, an advantage of the method described herein relates to the possibility of performing relevant method steps in situ.

The increased stability of the intermediates (such as compounds of formula (III)) against competing hydrolysis reactions, removes the need to work under anhydrous conditions. This makes it possible to avoid the use of hydrogensulfide gas, allowing instead the use of hydrosulfide salts, either as solids with crystal water or as solutions of the desired sulfide salt in water. Further it sets the stage for an in situ process where relevant steps may be performed in one-pot.

The present invention also discloses a novel process for the preparation of e.g. fluticasone propionate. By employing the method described herein, three out of five steps may be performed in one-pot, thus yielding fluticasone propionate in an overall yield of 89% (see example 3 herein).

Coupling agents and enhancers have primarily been used in peptide chemistry where the need to activate carboxylic acids in order to facilitate peptide couplings has been recognized for decades (Handbook of Reagents for Organic Synthesis, Activating Agents and Protecting Groups, ed. A. J. Pearson and W. R. Roush, John Wiley & Sons, 1999).

Within the field of steroids the use of coupling agents have also found use, especially in the preparation of carbothioic acids and esters of the androstane series (e.g. U.S. Pat. No. 4,188,385, U.S. Pat. No. 4,198,403, U.S. Pat. No. 4,335,121)). However, in these cases coupling enhancers were not used, and as a result some of the activated intermediates suffered the disadvantage of being prone to competing reactions, e.g. hydrolysis, resulting in a reduced yield of the final product. In U.S. Pat. No. 6,197,761, Glaxo made use of coupling enhancers. In example 16, a novel oxo-tetra-hydrofuranoyl amide was prepared by activating the androstane 17β-carboxylic acid with 1-hydroxybenzotriazole (HOBt) in conjunction with 1-ethyl-3-(3-dimethyl-aminopropyl) carbodiimide hydrochloride (EDC). However, it is not disclosed in this reference that the carbothioic acid can be obtained by adding a nucleophilic agent to an activated carboxylic group.

Embodiments of the present invention are described below, by way of examples only. The above mentioned references are incorporated by reference.

Definitions:

In the formulas, the substituents have the same meanings as in IUPAC Compendium of Chemical Terminology unless otherwise defined. When the substituent definition comprises a range (e.g. C6 to C22 or C1 to C10), the range is understood to comprise all integers in that range, i.e. 1, 2 3, 4, 5, 6, 7, 8, 9, 10, 11, 12, 13, 14, 15, 16, 17, 18, 19, 20, 21, 22 etc.

The term “substituted” means that one or more (such as 1, 2, 3, 4, 5, or 6) hydrogen atoms are substituted with substituents independently selected from groups such as: halogen atoms, nitro groups, hydroxyl, mercapto, cyano, carbamoyl, optionally substituted amino, optionally substituted alkyl (e.g. perhalogenalkyl), optionally substituted alkenyl, optionally substituted alkynyl, optionally substituted cycloalk(en/yn)yl, optionally substituted aryl, optionally substituted alkoxycarbonyl, optionally substituted aryloxycarbonyl, optionally substituted alkoxy, optionally substituted alkylthio, optionally substituted (hetero)aryl, optionally substituted (hetero)aryloxy or acyl groups. Two hydrogen atoms on the same carbon atom can be substituted with a divalent substituent, such as optionally substituted C1-C6 alkylene, O, NH, S.

The term “halogen” represents fluoro, chloro, bromo, or iodo.

The term “heteroatom” or “hetero” includes atoms such as O, S, or N.

The term “alkyl” includes straight or branched chain aliphatic hydrocarbon groups that are saturated and have 1 to 15 carbon atoms. Preferably, the alkyl group has 1-10 carbon atoms, and most preferred 1, 2, 3, 4, 5, or 6 carbon atoms. The alkyl groups may be interrupted by one or more heteroatoms, and may be substituted, e.g. with groups as defined above, such as halogen, hydroxyl, aryl, cycloalkyl, aryloxy, or alkoxy. Preferred straight or branched alkyl groups include methyl, ethyl, propyl, isopropyl, butyl and t-butyl. The term “alkoxy” stands for an —O-alkyl group.

The term “cycloalkyl” includes straight or branched chain, saturated or unsaturated aliphatic hydrocarbon groups which connect to form one or more rings of preferably 3, 4, 5, 6, or 7 ring members, which can be fused or isolated. The rings may be substituted, e.g. with groups as defined above, such as halogen, hydroxyl, aryl, aryloxy, alkoxy, or alkyl. Preferred cycloalkyl groups include cyclopropyl, cyclobutyl, cyclopentyl and cyclohexyl.

The term “alkenyl” includes straight or branched chain hydrocarbon groups having 2 to 15 carbon atoms (e.g. 2, 3, 4, 5, 6 or 10 carbon atoms) with at least one carbon-carbon double bond, the chain being optionally interrupted by one or more heteroatoms. The chain hydrogens may be substituted, e.g. with groups as defined above, such as halogen. Preferred straight or branched alkenyl groups include vinyl, allyl, 1-butenyl, 1-methyl propenyl and 4-pentenyl.

The term “alkynyl” includes straight or branched chain hydrocarbon groups having 2 to 15 carbon atoms (e.g. 2, 3, 4, 5, 6 or 10 carbon atoms) with at least one carbon-carbon triple bond, the chain being optionally interrupted by one or more heteroatoms. The chain hydrogens may be substituted, e.g. with groups as defined above, such as halogen. Preferred straight or branched alkynyl groups include ethynyl, propynyl, 1-butynyl, 1methyl propynyl and 4-pentynyl.

The term “cycloalkenyl” includes straight or branched chain, saturated or unsaturated aliphatic hydrocarbon groups which connect to form one or more non-aromatic rings of preferably 3, 4, 5, 6, or 7 ring members containing a carbon-carbon double bond, which can be fused or isolated. The rings may be substituted, e.g. with groups as defined above, such as halogen, hydroxyl, alkoxy, or alkyl. Preferred cycloalkenyl groups include cyclopentenyl and cyclohexenyl.

The term “aryl” refers to carbon-based rings which are aromatic. The rings may be isolated, such as phenyl, or fused, such as naphthyl. The ring hydrogens may be substituted, e.g. with groups as defined above, such as alkyl, halogen, free or functionalized hydroxy, trihalomethyl, etc. Preferred aryl groups include phenyl, 3(trifluoromethyl)phenyl, 3-chlorophenyl, and 4-fluorophenyl.

The term “heteroaryl” refers to aromatic hydrocarbon rings (having such as 3, 4, 5, 6, or 7 ring members) which contain at least one (e.g. 1, 2, 3, 4, or 5) heteroatom(s) in the ring. Heteroaryl rings may be isolated, preferably with 5 to 6 ring atoms, or fused, preferably with 8, 9 or 10 ring atoms. The heteroaryl ring(s) hydrogens or heteroatoms with open valency may be substituted, e.g. with groups as defined above, such as alkyl or halogen. Examples of heteroaryl groups include imidazole, pyridine, indole, quinoline, furane, thiophene, pyrrole, tetrahydroquinoline, dihydrobenzofuran, and dihydrobenzindole.

The term “aliphatic group” comprises both saturated and unsaturated, straight chain (i.e., unbranched), branched, cyclic, or polycyclic aliphatic hydrocarbons, which are optionally substituted with one or more functional groups. The term includes, but is not limited to, alkyl, alkenyl, alkynyl, cycloalkyl, cycloalkenyl, and cycloalkynyl moieties. It is presently preferred that alkyl or other aliphatic groups have 1-6 carbon atoms (which may be substituted or unsubstituted as specified). For example, suitable aliphatic groups include substituted or unsubstituted linear, branched or cyclic alkyl, alkenyl, alkynyl groups and hybrids thereof such as (cycloalkyl)alkyl, (cycloalkenyl)alkyl or (cycloalkyl)alkenyl.

The term “heteroaliphatic group” refers to aliphatic moieties (cf. the term aliphatic as defined above), which contain one or more oxygen, sulfur, nitrogen, phosphorous or silicon atoms, e.g., in place of carbon atoms. Heteroaliphatic moieties may be substituted or unsubstituted, branched, unbranched, cyclic or acyclic, and include saturated and unsaturated heterocycles such as morpholino, pyrrolidinyl, etc

The term “carbocyclic group/ring” includes a mono or bicyclic carbocyclic ring (e.g., cycloalkyl or cycloalkenyl groups such as cyclopropyl, cyclobutyl, cyclopentyl, cyclohexyl, cycloheptyl, cyclooctyl, cyclononyl, cyclopentenyl, cyclohexenyl, and bicyclo[2.2.1]heptanyl, bicyclo[3.2.1]octanyl and bicyclo[5.2.0]nonanyl, etc.); optionally containing 1-2 double bonds and optionally substituted by 1 to 3 suitable substituents as defined above.

The term “heterocyclic group/ring” includes both heteroaryl as above defined as well as non-aromatic ring systems having five to fourteen members, preferably five to ten, in which one or more ring carbons, preferably one to four, are each replaced by a heteroatom such as N, O, or S. Examples of heterocyclic rings include 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocyclic” whether saturated or partially unsaturated, also refers to rings that are optionally substituted with substituents as above defined.

The term “acyl” encompasses carboxylic acyl groups having the formula A-C(═O)—, in which formula A represents a substituent as defined above, such as an alkyl, alkenyl, aryl, heteroaryl or aralkyl group, the chain in said groups being optionally interrupted by one or more heteroatoms and the groups being optionally substituted, e.g. by one or more substituents as defined above. Examples on acyl groups are formyl, C1-C6 alk(en/yn)ylcarbonyl, arylcarbonyl, aryl-C1-C6 alk(en/yn)ylcarbonyl, cycloalkylcarbonyl, or cycloalkyl-C1-C6 alk(en/yn)ylcarbonyl group. Also, the term acyl comprises any of the above groups in which the C(═O) group is replaced by C(═S) or C(N—R), R is H or a substituent as defined above.

The term “hydroxy-protecting group” is intended to mean any group used for the temporary protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. Alkoxyalkyl protecting groups are groups such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals.

A “protected hydroxy” group is a hydroxy group derivatised or protected by any of the above groups commonly used for the temporary or permanent protection of hydroxy functions, e.g. the silyl, alkoxyalkyl, acyl or alkoxycarbonyl groups, as previously defined

The term “steroid” as used herein is intended to mean compounds having a cyclopentanophenanthrene nucleus. In these structures, the use of bold and dashed lines to denote particular conformation of groups again follows the IUPAC steroid-naming convention. The symbols “alpha” and “beta” indicate the specific stereochemical configuration of a substituent at an asymmetric carbon atom in a chemical structure as drawn. Thus “alpha.” denoted by a broken line, indicates that the group in question is below the general plane of the molecule as drawn, and “beta” denoted by a bold line, indicates that the group at the position in question is above the general plane of the molecule as drawn.

The terms “steroidal carbothioate”, “steroidal carbothioic acid” and “steroidal carboxylic acid” are intended to mean compounds in which a carbothioate group, carbothioic acid group or carboxylic acid group, respectively, is bound to the steroid nucleus, either directly or via linker, such as an optionally substituted C1-C6 alkylene group, in any position of the nucleus. Presently preferred are steroidal compound where the carbothioate group, carbothioic acid group or carboxylic acid group is bound to the carbon atom in position 17, more preferably directly to the nucleus without an intervening linker, and most preferably in beta configuration.

The term “carbothioate” is intended to mean the substituent —C(═O)—S—R, wherein R represents a substituent selected from: an aliphatic group, an heteroaliphatic group, a carbocyclic group, a heterocyclic group, a heteroaryl group, an aryl group, etc; all said groups are optionally substituted as above defined. Presently preferred R is substituted alkyl or substituted heterocyclyl.

The term “nucleophilic agent” means a chemical compound capable of forming a covalent bond with an activated carboxylic acid group.

The term “electrophilic agent” as used herein relates to a chemical compound capable of forming a covalent bond with a electron rich system such as e.g. a thioanion (—S⁻). Examples are compounds of the formula X—Y, where X is halogen and Y is aryl, a heterocyclic group or an aliphatic or heteroaliphatic group, said groups being optionally substituted. Presently, preferred agents are optionally substituted alkylhalogenides or optionally substituted heterocyclylhalides.

The term “solvate” represents an aggregate that comprises one or more molecules of the compound of the invention, with one or more molecules of solvent. Solvents may be, by way of example, water, ethanol, acetone, THF, DMA, or DMF.

### DETAILED DESCRIPTION OF THE INVENTION

A first aspect of the invention relates to a method for preparing a steroidal carbothioic acid or a salt thereof, said method comprises;

A) reacting a steroidal carboxylic acid or a salt thereof with a coupling agent [such as a carbodiimide derivative] alone or in conjunction with a coupling enhancer [such as a chemical entity comprising a saturated or unsaturated 5-6 membered heterocyclic ring in which the 5-6 membered heterocyclic ring contains one, two or three nitrogen atoms, said heterocyclic ring is optionally substituted on at least one nitrogen atom (such as by a hydroxy- or a cyano group) and/or optionally substituted on at least one carbon atom (such as by a keto group, a sulfonic acid or a salt thereof (i.e. the substituent is a sulfonate group, —S(═O)(═O)—O⁻ wherein the single bonded oxygen is bond directly to hydrogen, or to a group IA or IIA light metal (such as sulfonic acid sodium salt) or to an optionally substituted ammonium), an aliphatic group (e.g. alkyl comprising 1-4 carbon atoms, such as a methyl group) or a heteroaliphatic group preferably comprising a sulfur atom (e.g. heteroalkyl with 1-4 carbon atoms, such as a thiomethyl group)) and, when the said heterocyclic ring contains two adjacent carbon atoms, the said ring is optionally fused with an aromatic- or a heteroaromatic ring which is optionally substituted (such as by a halogen atom e.g. a chloro atom)]; and

B) reacting the product of step A) with a nucleophilic agent comprising a sulfur atom [such as hydrogen sulfide or a corresponding salt, optionally a hydrated salt thereof and preferably sodium hydrosulfide hydrate].

As coupling agent, it is presently preferred to use a carbodiimide derivative, represented by the following formula: Rₐ—N═C═N—R_{b}

wherein Rₐ and R_{b} are the same or different, and each represent an aliphatic, heteroaliphatic, carbocyclic or a heterocyclic group [all said groups are optionally sustituted]; more preferably the coupling agent is 1-ethyl-3-(3-dimethylaminopropyl) carbodiimide (EDC); and most preferably the hydrochloride salt of EDC. But the coupling agent can also be selected from the group consisting of:

A) derivatives of guanidinium N-oxide salts (N-methyl methanaminium salts) of a unsaturated 5-membered heterocyclic ring fused to an optionally substituted aryl, heteroaryl, benzene- or pyridine ring, (such as compounds of formula (A)), 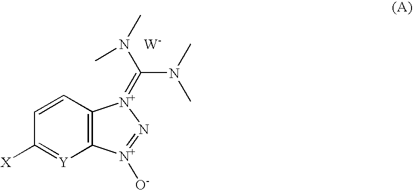
X=H, F, Cl, Br and Y═CH, N, O, S, W⁻═PF₆, BF₄, SbCl₆;

B) derivatives of uronium salts (O-hydronated ureas) of a unsaturated 5-membered heterocyclic ring fused to a optionally substituted aryl, heteroaryl, benzene- or pyridine ring, (such as compounds of formula (B)), 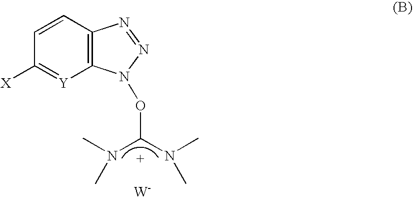
X=H, F, Cl, Br and Y═CH, N, O, S, W⁻═PF₆, BF₄, SbCl₆;
and;

C) derivatives of thiouronium salts (such as compounds of formula (C), preferably as the tetrafluoroborate salt), 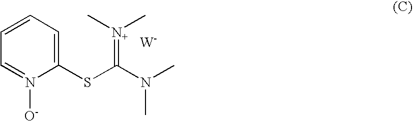
W⁻═BF₄, PF₆, SbCl₆

In an embodiment of the invention, the coupling enhancer is selected from the group consisting of:

A) a heterocyclic ring containing one or two nitrogen atoms, said ring being optionally substituted [such as by a keto group, a hydroxy group, an aliphatic group, a heteroaliphatic group, a cyano group, a halogen atom]; such as a compound of formula (D) or formula (E), 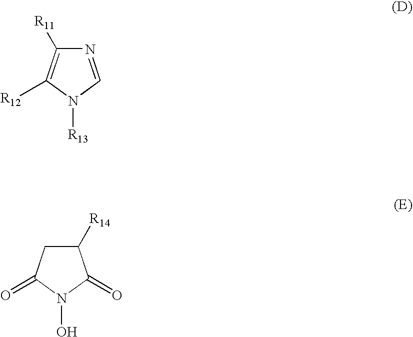
wherein R₁₁ and R₁₂ can be the same or different, and each represent a hydrogen atom or a cyano group (C≡N); R₁₃ represent a hydrogen atom or an alkyl group (such as methyl); and R₁₄ represent a hydrogen atom or a salt of a sulfonic acid such as sodium sulfonate [—S(═O)(═O)—O—O⁻Na⁺]; and

B) an unsaturated 5-6 membered heterocyclic ring fused to an aromatic- or heteroaromatic ring in which the said heterocyclic ring contains three nitrogen atoms, said rings being optionally substituted [such as with a hydroxy group, a halogen atom, a keto group and/or an optionally substituted aliphatic- or heteroaliphatic group], such as a compound of formulas (F), (G) 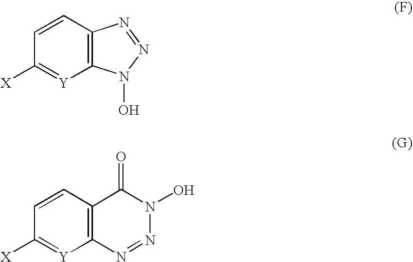
X=H, F, Cl, Br and Y═CH, N, O, S
preferably 6-chloro-hydroxybenzotriasole (6-Cl-HOBt), 7-aza-hydroxybenzotriasole (HOAt), or 3-hydroxy-4-oxo-3,4-dihydro-1,2,3-benzotriazine (Dbht-OH).

In a further embodiment of the invention, the nucleophilic agent comprising a sulfur atom is selected from the group comprising:
compounds of formula [M]⁺[SH]⁻ wherein M is a metal such as Li, Na or K; or [M]²⁺[S]²⁻ wherein M is a metal such as Ca or Mg, the said sulfide salts being optionally hydrated (such as sodium hydrosulfide hydrate); and
an in situ generated sulfide salt or a hydrated sulfide salt.

The nucleophilic agent can be added in the form of a solid salt or dissolved in a suitable solvent prior to addition to the reaction mixture, eg, as a solution of the salt in water and/or an organic solvent or a combination thereof.

In a presently preferred embodiment, the invention relates to preparing a steroidal carbothioic acid of formula (IV) or a salt thereof;

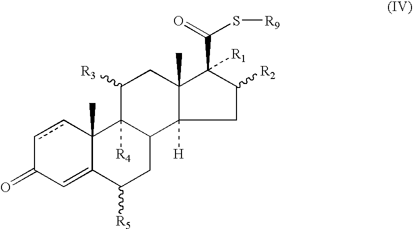

The symbol [symbol] in the 1,2-position represent a single or a carbon-carbon double bond.

R₁ represents a hydrogen atom, a hydroxy- or an alkoxy group (such as optionally substituted C₁₋₆ alkoxy) in the α-configuration, a group —O—C—(═O)—R₆, where R₆ is an alkyl group (such as optionally substituted C1-6 alkyl) or an optionally substituted 5-6 membered heterocyclic ring containing either oxygen, nitrogen or sulfur as ring hetero atom [such as a furanyl-, pyrrolyl- or thiophenyl group];

R₂ represents a hydrogen atom, a hydroxy group, an alkoxy group (such as optionally substituted C₁₋₆ alkoxy) in the α-configuration, an alkyl group (such as optionally substituted C₁₋₆ alkyl) which may be in either the α- or β-configuration, an alkylene group (such as optionally substituted C₁₋₆ alkylene having the two free valencies on the same carbon atom, preferably methylen) [the alkylene group is bound to the steroid nucleus via a double bond] or R₁ and R₂ together represent 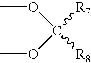
where R₇ and R₈ are the same or different and each represent a hydrogen atom or an alkyl group (such as optionally substituted C₁₋₆ alkyl);

R₃ represent hydrogen, hydroxy or a protected hydroxy group in either the α- or β-configuration or an oxo group (in which case the bond between R₃ and the steroid nucleus is a double bond);

R₄ represents a hydrogen atom or a halogen atom or R₃ and R₄ together represent a carbon-carbon bond or an epoxy group in the β-configuration; and

R₅ represents a hydrogen atom or a halogen atom in either the α- or β-configuration;

R₉ represents a hydrogen atom or R₉ represent a metal ion [eg. the moiety —S—R₉ represents a group of the formula [—S]⁻[M]⁺ wherein M is Li, Na or K]; the method comprising;

A) reacting a steroidal carboxylic acid of formula (II) 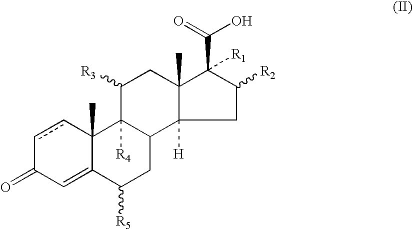
in which the substituents of formula (II) have the above defined meaning, or a salt thereof, with an coupling agent alone or in conjunction with an coupling enhancer, followed by the reaction with a nucleophilic agent comprising a sulfur atom; and optionally

B) reacting the product from step A) with an acid.

The sequence of addition of the coupling agent and the coupling enhancer is not considered to be very important, as the agent can be added before the enhancer, or vice versa. It is also possible to add a mixture of a steroidal carboxylic acid to a mixture of the agent and the enhancer or vice versa. Presently, it is preferred to add the agent and the enhancer, in succession, as solids to a steroidal carboxylic acid dissolved in a polar aprotic solvent, preferably DMF or DMA, optionally at a elevated temperature

The carbothioic acid or a salt thereof can be used in a process for producing steroidal carbothioates, and therefore the invention in a second aspect relates to a method for preparing a steroidal carbothioate (i.e. the carbothioic ester of the steroid), or a salt thereof, the method comprising: reacting a steroidal carbothioic acid or a salt thereof, which is prepared as defined in the first aspect of the invention, with an electrophilic agent [such as a di-or trihaloalkane]. Preferably the electrophilic agent is selected from the group consisting of: C₁₋₆ di- or trihaloalkanes, preferably a trihalo- or a dihalomethane, such as chlorobromomethane or bromofluoromethane.

In a preferred embodiment, the invention relates to a method for preparing a steroidal carbothioate of formula (I)

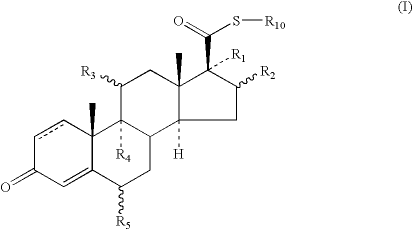

The symbol [symbol] in the 1,2-position represent a single or a carbon-carbon double bond.

wherein R₁, R₂, R₃, R₄, and R₅ are defined as above; and

R₁₀ represents a C₁₋₆ haloalkyl [such as a fluoro-, chloro-, bromomethyl group, a difluoromethyl or a trifluoromethyl group, or a 2′-fluoroethyl group] or a optionally substituted heterocyclic ring [such as a tetrahydrofuranyl group, preferably a 2-oxo-tetrahydrofuran-3-yl], the method comprising:

A) reacting a steroidal carboxylic acid of formula (II) 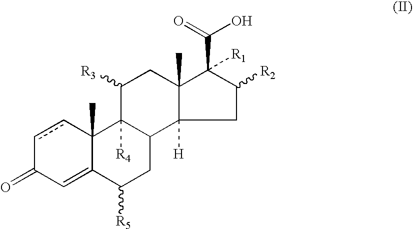
with a coupling agent [such as a carbodiimide] and a coupling enhancer [such as a compound of formula (D) or formula (E) 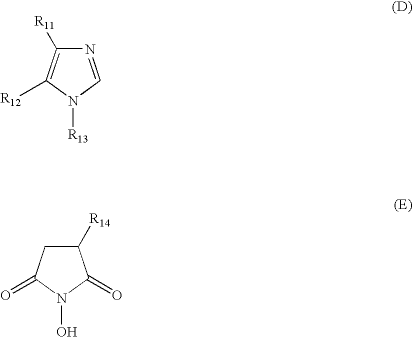
wherein R₁₁ and R₁₂ independently represent a hydrogen atoms or a cyano group (C≡N);

R₁₃ represent a hydrogen atom or an alkyl group (such as methyl); and

R₁₄ represent a hydrogen atom or a moiety of a sulfonic acid, such as sodium sulfonate (eg. the group —S(═O)(═O)—O⁻Na⁺)];

B) reacting the product from step A) with a nucleophilic agent comprising sulfur; and

C) reacting the product from step B) with an electrophilic agent [such as a C₁₋₆ di- or trihaloalkane, preferably a trihalo- or a dihalomethane such as chlorofluoromethane or bromofluoromethane] or a compound of the following formula; 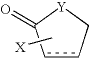
wherein X=H, F, Cl, Br and; Y═CH₂, NH, O, S, preferably X=Cl and Y═O.

In this method it is preferred that formula (D) is NMI (N-methylimidazole) or DCI (4,5-dicyanoimidazole), or formula (E) is NHS (N-hydroxysuccinimide) or sulfo-NHS (a N-hydroxysulfosuccinimide salt).

In a further embodiment, the invention relates to the above method wherein at least one of R₁₁ and R₁₂ is a cyano group (C≡N), and/or R₁₃ is a hydrogen atom, and/or R₁₀ is a fluoromethyl group.

In a third embodiment, step C) constitutes the in situ reaction of the product from step B) with bromofluoromethane to form a compound of formula (I) wherein R₁₀ is a fluoromethyl group, such as fluticasone propionate.

It is presently preferred that the method is performed without unnecessary isolation of intermediates, and thus in a preferred embodiment, at least two subsequent steps of the method are performed in situ, i.e. without any change or removal of solvents, or isolation of the individual intermediates. Advantageously, the steps A), B) and C) are conducted as a one-pot synthesis without solvent changes and/or are performed at room or elevated temperature. Also, the method can be conducted as a continuous method.

In an interesting embodiment, the invention relates to a method, wherein an androstane 17β-carboxylic acid is converted to an androstane 17β-carbothioate. In an other interesting embodiment, step B) provides an alkalimetal salt of the thioic acid, such as a compound of formula (IV), in which the moiety —S—R₉ represent a group of the formula [—S]⁻[M]⁺ wherein M is Li, Na or K e.g. —S⁻ Na⁺, and the other substituents have the same meaning as defined for formula (I).

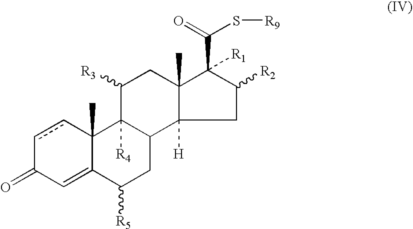

A third aspect of the invention relates to a method for producing a compound of the formula

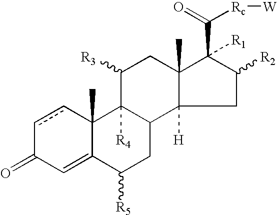

The symbol [symbol] in the 1,2-position represent a single or a carbon-carbon double bond Wherein wherein R₁, R₂, R₃, R₄, and R₅ are defined as above;

R_{c} represents S, O, NH; and

W represents hydrogen, an optionally substituted aliphatic- or heteroaliphatic group [such as a C₁₋₆ alkyl group substituted with one or more halogens] or an optionally substituted [such as with ═O] heterocyclic ring containing either O, N or S as ring hetero atom [such as a tetrahydrofuranyl-(lactone), tetrahydrothiophenyl-(thiolactone) or a pyrrolidinyl (lactame) group] which method comprises reacting a corresponding compound wherein Rc represents O and W represents H or a salt thereof with a coupling agent alone or in conjunction with a coupling enhancer, (the agent and enhacer defined in claim 1, step A); and reacting the resulting product with a nucleophilic agent having the formula: Rd-W, wherein W is defined as above and Rd represents OH, NH₂ or SH, or a salt thereof.

A fourth aspect of the invention relates to the novel products which can be obtained by the methods of the invention, or novel compounds which are intermediates in the methods. Thus, the invention relates to a compound of the formula (III) and salts and solvates thereof

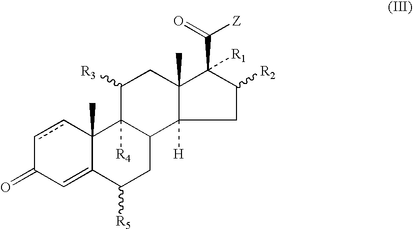

The symbol [symbol] in the 1,2-position represent a single or a carbon-carbon double bond. wherein R₁, R₂, R₃, R₄, and R₅ are defined as above; and

Z represent the structural moiety resulting from the reaction between the steroidal carboxylic acid of formula (II) and a coupling agent (preferably EDC), followed by a coupling enhancer as defined above, such as a compound selected from the group consisting of the compounds of formulas (D); (E); (F); and (G): 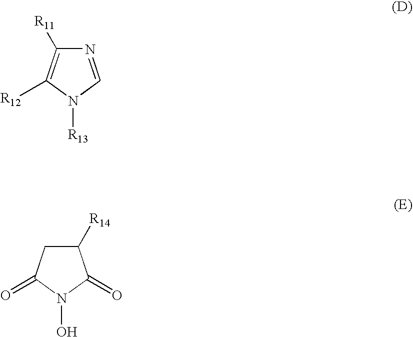
wherein R₁₁ and R₁₂ represent a hydrogen atom or a cyano group; R₁₃ represent a hydrogen atom or a alkyl group (such as methyl); and R₁₄ represent a hydrogen atom or a moiety of a sulfonic acid, such as sodium sulfonate [ie. the group —S(═O)(═O)—O⁻ Na⁺] 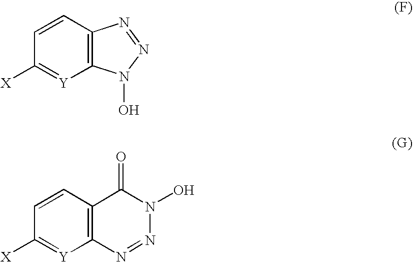
X=H, F, Cl, Br and Y═CH, N, O, S
with the proviso that 1-[(9alpha-fluoro-11beta-hydroxy-16beta-methyl-3-oxo-17alpha-propionyloxyandrosta-1,4-dien-17beta-yl)carbonyl]imidazole is disclaimed. This compound is mentioned in Phillips et al. (J. Med. Chem., 1994, 37, 3717-3729)

The compounds of the invention also comprises salts and solvates of the above formulas, and the skilled person will know that the compounds exsist in several polymorph forms, which also is an aspect of the present invention. Also, the above methods lead to compounds in the form of free bases, salts and polymorphs.

Interesting compounds of formula III are compounds wherein at least one of R₁₁ and R₁₂ is a cyano group (C≡N), and/or compounds, wherein R₁₃ is a hydrogen atom, and/or compounds obtained using a coupling enhancer selected from the group consisting of: NMI (N-methylimidazole); DCI (4,5-dicyanoimidazole); NHS (N-hydroxysuccinimide); and sulfo-NHS (N-hydroxysulfosuccinimide sodium salt) and/or
a compound having the formula:

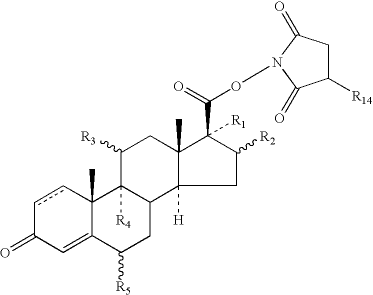

The symbol [symbol] in the 1,2-position represent a single or a carbon-carbon double bond.

in which the substituents have the same meaning as defined above, and salts and solvates thereof.

The fifth aspect of the invention relates to a composition comprising a compound according to the invention, and, as a sixth aspect, the use of a compound of the invention as an intermediate in a method for preparing a steroidal carbothioate or a steroidal carbothioic acid.

In a very interesting embodiment, the invention relates to the use of a compound of the invention as an intermediate in a method for preparing fluticasone propionate, especially in a method which comprises reaction with a nucleophilic agent comprising a sulfur atom [such as sodium hydrosulfide hydrate] and/or comprises reaction with an electrophilic agent [such as a C₁₋₆ di- or trihaloalkane, preferably a trihalo- or a dihalomethane such as chlorofluoromethane or bromofluoromethane].

The method as described herein relates in a presently preferred embodiment to the conversion of androstane 17β-carboxylic acids to 17β-carbothioates such as e.g. fluticasone propionate via novel in situ activated 17β-carboxylic acid intermediates. In a preferred embodiment of the method the 17β-carbothioic esters are prepared by reacting an acid of formula (II) with a coupling agent in conjunction with a coupling enhancer. The terms “coupling agent” and “coupling enhancer” are herein used to refer to chemical reagents that facilitate the formation of a reactive intermediate of formula (III) where Z represent a group with the structural formula (D) or formula (E). Such intermediates may be formed between androstane 17β-carboxylic acids and a carbodiimide derivative such as 1-ethyl-3-3-dimethylaminopropyl) carbodiimide hydrochloride (EDC) (a preferred example of a coupling agent) in combination with suitable coupling enhancers. Suitable coupling enhancers as used herein can for example be represented by the structures of formula (D) and formula (E) as described above.

The coupling agent and the optional coupling enhancers are preferably water-soluble so the reactions could be effected in aqueous solutions if preferred.

### PRESENTLY CONSIDERED THE BEST MODE OF THE INVENTION

Compounds of formula (III) as defined hereinbefore may be prepared by treating a compound of formula (II) with EDC in combination with a compound of formula (D). The resulting 17β-carboxy imidazolyl ester, which may be isolated if required, is treated with a nucleophilic agent (a sulfur source) that displaces the active ester group to form a compound of formula (IV), In one embodiment of the invention R₉ may represent optionally substituted C₁₋₆ alkyl groups like e.g. methyl, ethyl or a isopropyl group (preferably substituted with a halogen atom), or a 5-6 membered heterocyclic ring containing either oxygen, nitrogen or sulfur, the said heterocyclic ring being optionally substituted by a oxo group on either of the ring carbons. Compounds of formula (IV) may readily be isolated. In another embodiment of the invention, R₉ represent a hydrogen atom or a salt. Preferred examples of salts include alkali metal salts; e.g. Li, Na or K, alkaline earth metal salts; e.g. Ca or Mg, tertiary amine salts; e.g. pyridinium, triethylammonium or diisopropylethylamine salt, or quaternary ammonium salts; e.g. tetrabutylammonium salt. Such compounds of formula (IV) may be isolated if necessary. Subsequent addition of an electrophilic agent leads to the formation of compounds of formula (I) as depicted in scheme 1, wherein R₁₀ is a C₁₋₆ haloalkyl, a fluoro-, chloro-, or bromomethyl group, a difluoromethyl or a trifluoromethyl group, or a 2′-fluoroethyl group.

Preferably the nucleophilic agent is a hydrogen sulfide or a salt thereof or more preferably a hydrated sulfide salt such as sodium hydrosulfide hydrate.

Preferably the electrophilic agent is chlorofluoromethane or more preferably bromofluoromethane.

In a preferred embodiment of the present method, compounds of formula (III) as defined hereinbefore may be prepared by treating a compound of formula (II) with EDC in combination with a compound of formula (E). The resulting 17β-carboxy succinimidyl ester which may be isolated if required is treated with a nucleophilic agent that displaces the active ester group to form a compound of formula (IV) as defined hereinbefore. Subsequent addition of a electrophilic agent conclude the synthesis towards compounds of formula (I) as depicted in scheme 1, wherein R₁₀ is a C₁₋₆ haloalkyl, a fluoro-, chloro-, or bromomethyl group, a difluoromethyl or a trifluoromethyl group, or a 2′-fluoroethyl group. The formation of compounds of formula (I) may take place in a one-pot reaction starting with compounds of formula (II) without isolation of the intermediates of formula (II) and (IV) if e.g. sodium hydrosulfide hydrate is used as the nucleophilic agent.

The use of a coupling enhancer in combination with a carbodiimide forms an active ester intermediate of formula (III) with increased stability and reactivity. These intermediates have an increased threshold against competing hydrolysis reactions. The reduced level of hydrolysis raises the efficiency regarding the formation of products of formula (I) (scheme 1, see below) where R₁₀ is defined as hereinbefore.

Surprisingly, we have found that the use of these novel 17β-carboxy succinimidyl- and 17β-carboxy imidazolyl esters of formula (III) reduce the cycle time, increase the yield and enhance the purity of the final product of formula (I).

Compound Preparation

The method of the disclosed invention will be better understood when related to the following synthetic scheme, which illustrates a preferred embodiment of the method as described herein. Moreover, persons skilled in the art will be aware of variations of, and alternatives to the method described hereinafter. The provided examples will demonstrate the synthesis and allow compounds defined by formula (I) to be obtained. In scheme 1, the substituents have the above-defined meaning.

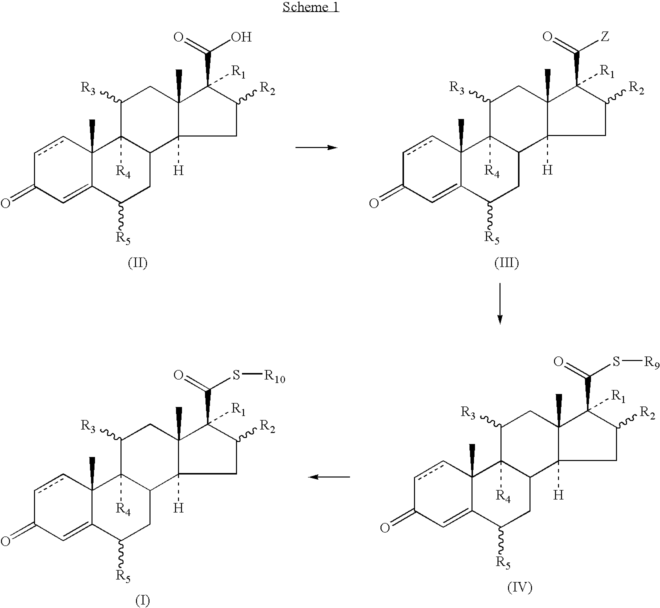

The androstane 17β-carboxylic acid starting materials employed in the present invention may be readily prepared by any means known in the art. Compounds of formula (II) can be prepared by oxidation of a suitable 21-hydroxy-20-keto pregnane of formula (V) as taught in e.g. U.S. Pat. No. 3,636,010 (example 1).

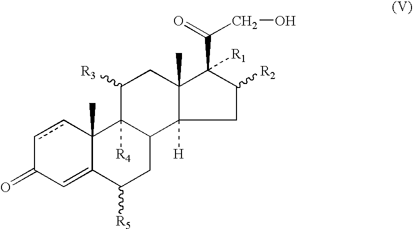

Compounds of formula (V) which are commercially available include e.g. budesonide, desonide, triamcinolone acetonide, fluocinolone acetonide, betamethasone, dexamethasone, prednisolone, flumethasone, beclomethasone, icomethasone, diflorasone, hydrocortisone and fludrocortisone.

The method as described herein will now be described in detail in connection with other particularly preferred embodiments of scheme 1. Thus, the following and not limiting examples will illustrate an especially preferred methodology. The examples are presented to provide what is believed to be the most useful and readily understood description of the procedures and conceptual aspects of the method described herein.

### EXAMPLES

General

MS (ESI) refer to mass spectra obtained using electrospray ionisation techniques. Melting points were obtained using a Mettler FP81 MBC cell and are uncorrected. The purity of the products was determined by RP18-HPLC analysis using UV-detection at 239 nm.

Abbreviations; DMF (N,N-dimethylformamide), DMA (N,N-dimethylacetamide), TMF (tetrahydrofurane), EDC (1ethyl-3-(3-dimethyl-aminopropyl)carbodiimide hydrochloride), NHS (N-hydroxysuccinimide), NMI (N-methylimidazole) and DCI (4,5-dicyano-imidazole).

### Example 1

### 6α,9α-difluoro-11β,17α-dihydroxy-16α-methyl-3-oxoandrosta-1,4-diene-17β-carboxylic acid

6α,9α-difluoro-11β,17α-21-trihydroxy-16α-methyl-pregna-1,4-diene-3,20-dione (flumetasone) (5.25 g, 12.78 mmol) was dissolved in THF (25 ml) at 0° C. To the stirred solution, periodic acid (2.0 eq., 5.83 g in 12 ml H₂O) was added drop wise. The reaction flask was covered in aluminum foil after 75 minutes and left stirring at room temperature. The reaction was quenched after 4.5 hours by addition of water (120 ml) and left in a refrigerator over-night. The resulting precipitate was filtered, washed with water until the filtrate was neutral to pH-paper and dried under reduced pressure. The title compound was obtained as a white solid (4.96 g, 98%), with a purity of >99% by HPLC.

### Example 2

### 6α,9α-difluoro-11β-hydroxy-16α-methyl-3-oxo-17α-propionyloxyandrosta-1,4-diene-17β-carboxylic acid

A suspension of 6α,9α-difluoro-11β,17α-dihydroxy-16α-methyl-3-oxoandrosta-1,4-diene-17β-carboxylic acid (4.11 g, 10.38 mmol) in acetone (30 ml) was added Et₃N (3.5 eq., 3.7 ml) under N₂ and stirring. The resulting solution was cooled to 0° C. and propionyl chloride (4.5 eq., 4.3 ml) was added drop wise to a white suspension. After 85 minutes Et₂NH (10.0 eq., 7.6 ml) was added and the resulting mixture was left stirring for 55 minutes at 0° C., before the solvent was removed under reduced pressure. The resulting solid was dissolved in water (20 ml), and treated with 1N HCl to pH 2. The white precipitate thus formed was filtered, washed with ice cold water and dried under reduced pressure at 60° C. The title compound was obtained as a white solid (4.64 g, 99%) with a purity of 99% by HPLC.

### Example 3

### S-fluoromethyl 6α,9α-difluoro-11β-hydroxy-16α-methyl-3-oxo-17α-propionyloxyandrosta-1,4-diene-17β-carbothioate (fluticasone propionate)

A flask charged with 6α,9α-difluoro-11β-hydroxy-16α-methyl-3-oxo-17α-propionyloxyandrosta-1,4-diene-17β-carboxylic acid (0.30 g, 0.66 mmol) in DMA (30 ml) at ˜50° C. was added EDC (3.0 eq., 0.38 g) and NHS (3.1 eq., 0.24 g) and left stirring until the reaction was complete, as monitored by HPLC analysis. The oil bath was turned off and sodium hydrosulfide hydrate (20.0 eq., 0.74 g) was added in one portion at ˜35° C. The addition immediately resulted in a strongly blue colored reaction mixture. After 15 minutes the reaction was complete and gaseous bromofluormethane was added at room temperature. The initial blue color gradually changed to pale yellow during the addition of bromofluoromethane. On completion of the reaction, after 15 minutes, as monitored by HPLC analysis, sodiumcarbonate (25 ml, 10% in water) followed by water (10 ml) was added to the cooled reaction mixture. The solid thus formed was filtered, dissolved in methanol (40 ml), heated to reflux and filtered. Temperated water (40 ml) was slowly added to the warm filtrate. The beginning suspension was left at room temperature for two hours before cooling in a refrigerator. The resulting precipitate was collected by filtration, washed with water (40 ml) and dried under reduced pressure. The title compound was obtained as a white amorphous solid (0.25 g, 92%) with a melting point of 267° C. and a purity of >98% by HPLC. Spiking of the product sample with the British Pharmacopoeia Standard (melting point: 265° C.) of fluticasone propionate, showed one peak by HPLC analysis.

### Example 4

### 6α,9α-difluoro-11β-hydroxy-16α-methyl-3-oxo-17α-propionyloxyandrosta-1,4-diene-17β-carbothioic acid

A flask charged with 6α,9α-difluoro-11β-hydroxy-16α-methyl-3-oxo-17α-propionyl-oxyandrosta-1,4-diene-17β-carboxylic acid (0.30 g, 0.66 mmol) in DMA (30 ml) was heated to ˜50° C. EDC (3.0 eq., 0.38 g) and NMI (3.1 eq., 162 μl) was added and the reaction mixture was left stirring until the reaction was complete, as monitored by HPLC analysis. The oil bath was turned off and sodium hydrosulfide hydrate (20.0 eq., 0.74 g) was added in one portion at ˜35° C. The addition immediately resulted in a strongly blue colored reaction mixture with some precipitation. After 15 minutes the reaction was complete and crushed ice followed by 2N HCl (10 ml) to pH ˜2 was added and the flask was left in a refrigerator. During the addition of HCl, the initial bluish reaction mixture gradually became colorless. The solid thus formed was filtered and a minimum amount of acetone following temperated water was added to a beginning crystallisation. The refrigerated suspension was filtered, washed with water (30 ml) until the filtrate was neutral to pH-paper and dried under reduced pressure. The title compound was obtained as a white amorphous solid (0.10 g, 32%) with a purity of 98% by HPLC.

### Example 5

### S-methyl 6α,9α-difluoro-11β-hydroxy-16α-methyl-3-oxo-17α-propionyloxyandrosta-1,4-diene-17β-carbothioate

6α,9α-difluoro-11β-hydroxy-16α-methyl-3-oxo-17α-propionyloxyandrosta-1,4-diene-17β-carboxylic acid (0.20 g, 0.43 mmol) was dissolved in DMF (10 ml). The reaction flask was immersed in an oil-bath and the temperature was raised to ˜40° C. EDC (˜2 eq., 0.15 g) and NHS (˜5 eq., 0.26 g) was added and the reaction mixture was left stirring at 40° C. for 19 hours. The temperature was raised to 50° C. and another portion of EDC (˜4 eq., 0.29 g) was added. The mixture was left stirring at 50° C. for 3 hours. The flask was removed from the oil-bath and the reaction mixture was diluted by the addition of DMF (5 ml). Aqueous NaSMe (1.0 ml, 21% solution in water) was added in portions over a period of 30 minutes. The reaction mixture turned into a pale pink suspension. The reaction was quenched by the addition crushed ice and aqueous HCl (12 ml, 10%). The white precipitate was filtered, washed with water and dried. The crude product was dissolved in a minimum amount of acetone and water was added until a beginning precipitation. The resulting solid was collected by filtration, washed with water and dried under reduced pressure to yield the title compound as a white solid (0.11 g, 51%) with a purity of 90% by HPLC.

### Example 6

### S-methyl 6α,9α-difluoro-11β-hydroxy-16α-methyl-3-oxo-17α-propionyloxyandrosta-1,4-diene-17β-carbothioate

Charge a flask with 6α,9α-difluoro-11β-hydroxy-16α-methyl-3-oxo-17α-propionyl-oxyandrosta-1,4-diene-17β-carboxylic acid (0.30 g, 0.66 mmol) in DMA (30 ml), and heat the contents to ˜50° C. Add EDC (3.0 eq., 0.38 g) and NMI (3.1 eq., 162 μl) and leave the mixture stirring until the reaction is complete, as monitored by HPLC analysis. Turn off/remove the oil bath and add aqueous NaSMe (1.5 ml, 21% solution in water) in one portion at ˜35 ° C. The reaction is quenched by the addition of crushed ice and aqueous HCl (12 ml, 10%). The white precipitate is filtered, washed with water and dried. The crude product is dissolved in a minimum amount of acetone and water is added to induce precipitation. The resulting solid is collected by filtration, washed with water and dried under reduced pressure to yield the title compound as a solid.

### Example 7

### S-fluoromethyl 6α,9α-difluoro-11β-hydroxy-16α-methyl-3-oxo-17α-propionyloxyandrosta-1,4-diene-17β-carbothioate (fluticasone propionate)

Charge a flask with 6α,9α-difluoro-11β-hydroxy-16α-methyl-3-oxo-17α-propionyl-oxyandrosta-1,4-diene-17β-carboxylic acid (0.30 g, 0.66 mmol) in DMA (30 ml) and heat the contents to ˜50° C. Added EDC (3.0 eq., 0.38 g) and DCI (3.1 eq.) and leave the mixture stirring until the reaction is complete, as monitored by HPLC analysis. Turn off/remove the oil bath and add sodium hydrosulfide hydrate (20.0 eq., 0.74 g) in one portion at ˜35 ° C. After x minutes the reaction is complete and gaseous bromofluoromethane is added at room temperature. Upon completion of the reaction, as monitored by HPLC analysis, sodium carbonate (25 ml, 10% in water) and water (10 ml) is added to the refrigerated reaction mixture. The solid which forms is filtered and dissolved in methanol (40 ml). The methanolic solution is heated to reflux and filtered. Temperate water (40 ml) is added slowly to the warm filtrate. The resulting suspension is left at room temperature for two hours before being transferred to a refrigerator. The precipitate which forms is collected by filtration, washed with water (40 ml) and dried under reduced pressure. The title compound is obtained as a solid.

1. A method for preparing a steroidal carbothiolic acid or a salt thereof, said method comprises:
A) reacting a steroidal carboxylic acid or a salt thereof with a coupling agent selected from the group consisting of carbodiimide derivatives represented by the following formula:
Rₐ—N═C═N—R_{b}
wherein Rₐ and R_{b} are the same or different, and each represent an aliphatic, heteroaliphatic, carbocyclic or a heterocyclic group, wherein the group is optionally substituted;
alone or in conjunction with a coupling enhancer; and
B) reacting the product of step A) with a nucleophilic agent comprising a sulfur atom.

2. A method according to claim 1 in which the coupling agent is 1-ethyl-3-(3-dimethylaminopropyl) carbodiimide (EDC).

3. A method according to claim 2, in which the coupling agent is the hydrochloride salt of EDC.

4. A method according to claim 1, in which the coupling enhancer is selected from the group consisting of:
A) a heterocyclic ring of formula (D) or formula (E), 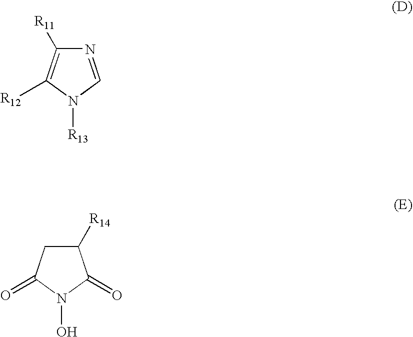
wherein R₁₁ and R₁₂ can be the same or different, and each represent a hydrogen atom or a cyano group; R₁₃ represent a hydrogen atom or an alkyl group; and R₁₄ represent a hydrogen atom or a salt of a sulfonic acid; and
B) an unsaturated 5-6 membered heterocyclic ring of formula (F) or formula (G), 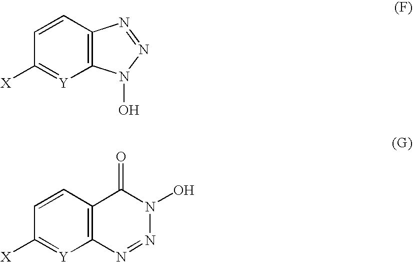
X=H, F, Cl, Br and Y═CH, N, O, S.

5. A method according to claim 1, where the nucleophilic agent comprising a sulfur atom is selected from the group consisting of:
compounds of formula [M]⁺[SH]⁻ wherein M is a metal such as Li, Na or K; or [M]²⁺[S]²⁻
wherein M is a metal such as Ca or Mg, the said sulfide salts being optionally hydrated; and
an in situ generated sulfide salt or a hydrated sulfide salt.

6. The method of claim 1, wherein the nucleophilic agent is dissolved in a suitable solvent prior to addition to the reaction mixture, or wherein the nucleophilic agent is added in the form of a solid salt or as a solution of the salt in water, an organic solvent, or a combination thereof.

7. A method according to claim 1 for preparing a steroidal carbothioic acid of formula (IV) or a salt thereof 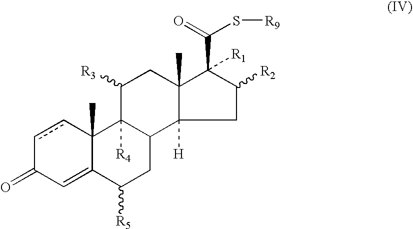
wherein the symbol [symbol] in the 1,2-position represent a single or a carbon-carbon double bond;
R₁ represents a hydrogen atom, a hydroxy- or an alkoxy group in the α-configuration, a group —O—C(═O)—R₆ is an alkyl group or an optionally substituted 5-6 membered heterocyclic ring containing either oxygen, nitrogen or sulfur as ring hetero atom;
R₂ represents a hydrogen atom, a hydroxy group, an alkoxy group in the n-configuration, an alkyl group which may be in either the η- or β-configuration, an alkylene group, wherein the alkylene group is bound to the steroid nucleus via a double bond, or R₁ and R₂ together represent 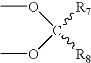
where R₇ and R₈ are the same or different and each represent a hydrogen atom or an alkyl group;
R₃ represent a hydrogen atom, hydroxy- or a protected hydroxy group in either a α- β-configuration or an oxo group;
R₄ represents a hydrogen- or a halogen atom or R₃ and R₄ together represent a carbon-carbon bond or an epoxy group in the βconfiguration; and
R₅ represents a hydrogen- or a halogen atom in either the α- or β-configuration;
R₉ represents a hydrogen atom or R₉ represent a metal ion; the method comprising;
A) reacting a steroidal carboxylic acid of formula (II) or a salt thereof 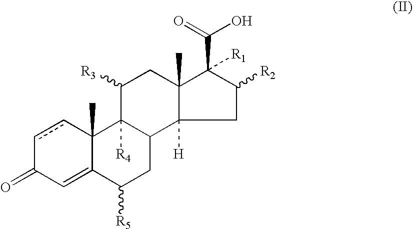
in which the substituents of formula (II) have the above defined meaning with a coupling agent alone or in conjunction with an coupling enhancer, followed by the reaction with a nucleophilic agent comprising a sulfur atom; and optionally
B) reacting the product from step A) with an acid.

8. The method of claim 1, wherein i)
the coupling agent is added before the coupling enhancer, or
the coupling enhancer is added before the coupling agent, and/or wherein ii)
the steroidal carboxylic acid is added to a mixture of the coupling agent and the coupling enhancer, or wherein
a mixture of the coupling agent and the coupling enhancer is added to a steroidal carboxylic acid, or wherein
the steroidal carboxylic acid is added to a mixture of the coupling agent and the coupling enhancer in a polar aprotic solvent, preferably DMF or DMA, at elevated temperature.

9. A method for preparing a steroidal carbothioate, or a salt thereof, the method comprising;
reacting a steroidal carbothioic acid or a salt thereof with an electrophillic agent.

10. A method according to claim 9, in which the electrophillic agent is selected from the group consisting of: C₁₋₈ di- or trihaloalkanes.

11. A method according to claim 9 for preparing a steroidal carbothioate of formula (I) 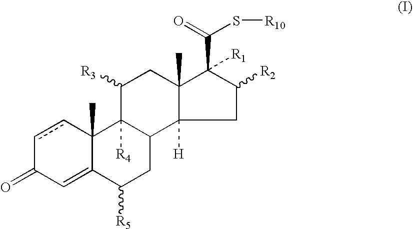
wherein R₁, R₂, R₃, R₄ and R₅;
R₁ represents a hydrogen atom, a hydroxy- or an alkoxy group in the α-configuration, a group —O—C(═O)—R₆ is an alkyl group or an optionally substituted 5-6-membered heterocyclic ring containing either oxygen, nitrogen or sulfur as ring hetero atom;
R₂ represents a hydrogen atom, a hydroxy group, an alkoxy group in the n-configuration, an alkyl group which may in either the η- or β-configuration, an alkylene group, wherein the alkylene group is bound to the steroid nucleus via a double bond, or R₁ or R₂ together represent 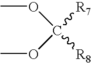
where R₇ and R₈ are the same or different and each represent a hydrogen atom or an alkyl group;
R₃ represent a hydrogen atom, hydroxy- or a protected hydroxy group in either a α- or β-configuration or an oxo group;
R₄ represents a hydrogen- or a halogen atom or R₃ and R₄ together represent a carbon-carbon bond or an epoxy group in the β-configuration; and
R₅ represents a hydrogen- or a halogen atom in either the α- or β-configuration
and R₁₀ represents a C₁₅ haloalkyl or an optionally substituted heterocyclic ring, the method comprising:
A) reacting a steroidal carboxylic acid of formula (II) 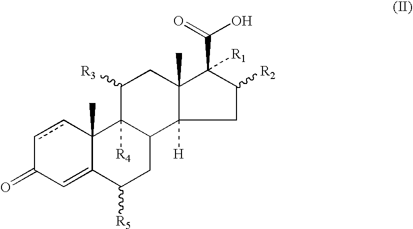
with a coupling agent and a coupling enhancer 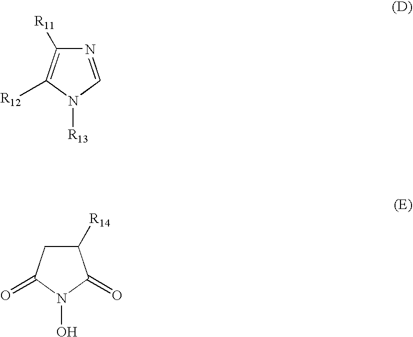
wherein R₁₁ and R₁₂ independently represent a hydrogen atom or a cyano group (C═N);
R₁₂ represent a hydrogen atom or an alkyl group; and
R₁₄ represent a hydrogen atom or a moiety of a sulfonic acid
B) reacting the product from step A) with a nucleophilic agent comprising sulfur; and
C) reacting the product from step B) with an electrophillic agent or a compound of the following formula; 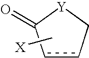
wherein X=H, F, Cl, or Br and; Y═CH₂, NH, O, or S.

12. The method of claim 11, wherein the coupling enhancer is selected from the group consisting of: NMI (N-methylimidazole); DCI (4,5-dicyanolmidazole); NHS (N-hydroxysuccinimide); and sulfo-NHS (N-hydroxysulfosuccinimide).

13. The method of claim 11, wherein step C) constitutes the in situ reaction of the product from step B) with bromofluoromethane to form a compound of formula (I) wherein R₁₀ is a fluoromethyl group.

14. The method according to claim 9, in which
at least two subsequent steps are performed in situ;
the method is conducted as a continuous method;
step A), B) and optionally step C) are conducted as a one-pot synthesis without solvent changes are performed at room or elevated temperature, or both; or
a combination of one or more of the foregoing.

15. The method of claim 9, wherein an androstane 17β-carboxylic acid is converted to an androstane 17β-carbothioate.

16. The method of claim 9, wherein step B) provides a compound of formula (IV), in which the moiety -5-R₅ represent a group of the formula [—S]⁻[M]⁺ wherein M is a metal such as Li, Na or K,
wherein the symbol [symbol] in the 1,2-position represent a single or a carbon-carbon double bone;
R₁ represents a hydrogen atom, a hydroxy- or an alkoxy group in the α-configuration, a group —O—C(═O)—R₆ is an alkyl group or an optionally substituted 5-6 membered heterocyclic ring containing either oxygen, nitrogen or sulfur as ring hetero atom;
R₂ represents a hydrogen atom, a hydroxy group, an alkoxy group in the n-configuration, an alkyl group which may be in either the η- or β-configuration, an alkylene group, wherein the alkylene group is bound to the steroid nucleus via a double bond, or R₁ or R₂ together represent
where R₇ and R₈ are the same or different and each represent a hydrogen atom or an alkyl group;
R₃ represent a hydrogen atom, hydroxy- or a protected hydroxy group in either a α- or β-configuration or an oxo group;
R₄ represents a hydrogen- or a halogen atom or R₃ and R₄ together represent a carbon-carbon bond or an epoxy group in the β-configuration; and
R₅ represents a hydrogen- or a halogen atom in either the α- or β-configuration;
R₉ represents a hydrogen atom or R₉ represent a metal ion.

17. A compound of the formula (III) and salts and solvates thereof 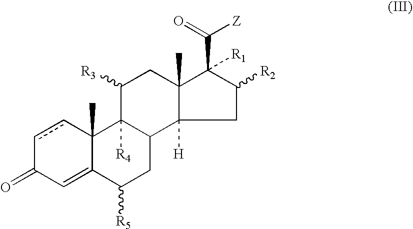
wherein R₁ represents a hydrogen atom, a hydroxy- or an alkoxy group in the α-configuration, a group —O—C(═O)—R₆ is an alkyl group or an optionally substituted 5-6-membered heterocyclic ring containing either oxygen, nitrogen or sulfur as ring hetero atom;
R₂ represents a hydrogen atom, a hydroxy group, an alkoxy group in the n-configuration, an alkyl group which may be in either the η- or β-configuration, an alkylene group, wherein the alkylene group is bound to the steroid nucleus via a double bond, or R₁ and R₂ together represent 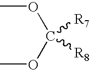
where R₇ and R₈ are the same or different and each represent a hydrogen tom or an alkyl group;
R₃ represent a hydrogen atom, hydroxy- or a protected hydroxy group in either a α- to β-configuration or an oxo group;
R₄ represents a hydrogen- or a halogen atom or R₃ and R₄ together represent a carbon-carbon bond or an epoxy group in the β-configuration; and
R₅ represents a hydrogen- or a halogen atom in either the α- or β-configuration; and
Z represent the structural moiety resulting from the reaction between the steroidal carboxylic acid of formula (II) and a coupling agent, followed by a coupling enhancer selected from the group consisting of the compounds of the formulas (D); (E); (F); and (G): 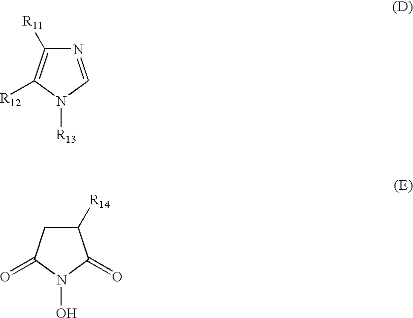
wherein R₁₁ and R₁₂ independently represent a hydrogen atom or a cyano group; R₁₃ represent a hydrogen atom or a methyl group; and R₁₄ represent a hydrogen atom or a moiety of a sulfonic acid, 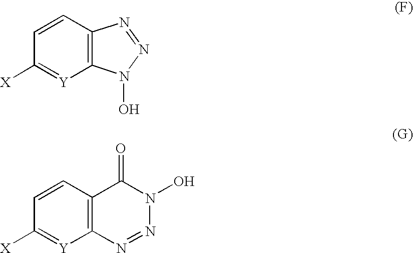
X=H, F, Cl, Br and Y═CH, N, O, S
with the proviso that:
when the coupling enhancer is a compound of formula (F), X can not represent H when Y represents CH:
when the coupling enhancer is a compound of formula (D), R₁₁ and R₁₂ can not both represent H when R₁ in formula III represents DH; and
when the coupling enhancer is a compound of formula (E), R₁₄ can not represent H when R₁ in formula III represents H;
and with the further proviso that
succinlmidyl-9υ-fluoro-11β, 17α-dihydroxy-16α-methyl-3-oxoandrosta-1,4-diene-17β-carboxylate;
17α-hydroxy-4-androsten-3-one-17β-carboxylic acid N-hydroxysuccinimide ester;
N-hydroxysuccinimidyl-9-fluoro-16α-methyl-11β,17-dihydroxy-3-oxo-1,4-androstadiene-17β-carboxyester;
N-hydroxysuccinimide ester of dexamethasone-17β-carboxylic acid; and 1-[(9-fluoro-11β-hydroxy-16β-methyl-3-oxo-17α-propionylaxyandrosta-1,4-dien-17β-yi)carbonyl]imidazol are disclaimed.

18. The compound of claim 17, wherein at least one of R₁₁ and R₁₂ is a cyano group (C═N), R₁₃ is a hydrogen atom, formula (D) is NMI (N-methylimidazole) or DCI (4,5-dicyano-imidazole), formula (E) is NHS (N-hydroxysuccinimide) or sulfo-NHS (N-hydroxysulfosuccinimide), or a combination comprising one or more of the foregoing.

19. The compound of claim 17, having the formula: 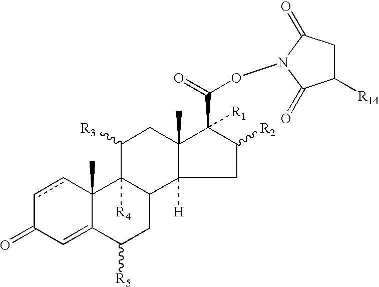
with the proviso that R₁₄ can not represent H when R₁ represents H.

20. A compound of the formula (VI) and salts and solvates thereof
wherein
R₁ represents a hydrogen atom, a hydroxy- or an alkoxy group in the α-configuration, a group —O—C(═O)—R₆ is an alkyl group or an optionally substituted 5-6 membered heterocyclic ring containing either oxygen, nitrogen or sulfur as ring hetero atom;
R₂ represents a hydrogen atom, a hydroxy group, an alkoxy group in the n-configuration, an alkyl group which may in either the η- or β-configuration, an alkylene group, wherein the alkylene group is bound to the steroid nucleus via a double bond, or R₁ and R₂ together represent 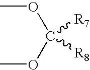
where R₇ and R₈ are the same or different and each represent a hydrogen tom or an alkyl group;
R₃ represent a hydrogen atom, hydroxy- or a protected hydroxy group in either a α- or β-configuration or an oxo group;
R₄ represents a hydrogen- or a halogen atom or R₃ and R₄ together represent a carbon-carbon bond or an epoxy group in the β-configuration; and
R₅ represents a hydrogen- or a halogen atom in either the α- or β-configuration,
wherein Rₐ and R_{b} are the same or different, and each represent an aliphatic, heteroaliphatic, carbocylic or a heterocyclic group;
with the proviso that 1-(3-dimethylamino-propyl)-3-ethyl-carbodiimide-6α,9υ-difluoro-11β-hydroxy-16α,17α-isopropylidenedioxy-3-oxo-androsta-1,4-diene-17β-carboxylate is disclaimed.

21-23. (canceled)
